(19) United States
(12) Patent Application Publication
(10) Pub. No.: US 2024/0226521 A1
(43) Pub. Date: Jul. 11, 2024

(54) TRANSDERMAL MICRONEEDLE PATCH CONTAINING POLY-LACTIC ACID NEEDLES THAT ARE EASILY ABSORBABLE INTO SKIN

(71) Applicant: SIMFLE STICK Co., Ltd., Daegu (KR)

(72) Inventor: Chang Hoon KIM, Daegu (KR)

(73) Assignee: SIMFLE STICK Co., Ltd., Daegu (KR)

(21) Appl. No.: 18/456,041

(22) Filed: Aug. 25, 2023

(30) Foreign Application Priority Data
Jan. 5, 2023 (KR) 10-2023-0001819

(51) Int. Cl. A61M 37/00

(52) U.S. Cl. CPC A61M 37/0015; A61M 2037/0023; A61M 2037/0053; A61M 2037/0061

(57) ABSTRACT

Proposed is a microneedle patch including at least one needle base disposed to protrude on a surface of the microneedle patch while having a patch layer as a flat layer, and made of hyaluronic acid (HA), and a needle tip disposed on an upper surface of the needle base, separated from the needle base by being inserted into a dermis layer of the skin, and made of the PLA, a biodegradable polymer. The PLA, a biodegradable polymer is characterized by after adding 0.01˜0.5 g of epidermal growth factor (EGF) to 0.1˜0.5 g of PLA and dissolving in 10 m [symbol] of dichloromethane (DCM) as an organic solvent, mixing same with 50 m [symbol] of 1 wt % poly vinyl alcohol (PVA) solution, and then mixing prepared PLA microparticles with a size of 30˜150 μm with 3 wt % carboxylic methyl cellulose (CMC) solution.

### CROSS REFERENCE TO RELATED APPLICATION

The present application claims priority to Korean Patent Application No. 10-2023-0001819, filed Jan. 5, 2023, the entire contents of which is incorporated herein for all purposes by this reference.

### BACKGROUND OF THE INVENTION

### Field of the Invention

The present disclosure relates to a transdermal microneedle patch containing microneedles that are easily absorbable into the skin and, more particularly, to a transdermal microneedle patch containing poly-lactic acid microneedles that are easily absorbable into the skin, in which poly-lactic acid (PLA), a biodegradable polymer, is combined with epidermal growth factor (EGF) and hyaluronic acid (HA) to manufacture a non-invasive microneedle adopted as a patch-type filler. According to the present disclosure, since only microneedles are inserted into the skin, use thereof does not require a high degree of skill, and use and convenience are improved. Safety is also improved as there is no secondary infection. In addition, the microneedle patch enables rapid healing with little tissue damage, and is patient-friendly because it does not cause pain. Furthermore, the PLA and EGF elements may be directly delivered into the living tissue after the microneedle patch is applied, and a sufficient amount of PLA and EGF elements may be delivered effectively, stimulating collagen regeneration in skin. Therefore, the application of a filler may be specialized according to cosmetic purposes, and the advantages of a microneedle patch-type filler that delivers substances through the skin may be cosmetically active maximized.

### Description of the Related Art

In general, a drug delivery system (DDS) refers to a series of techniques that deliver drugs to target sites such as cells and tissues to reduce side effects and maximize efficacy by controlling the absorption and release of drugs. The DDS includes oral administration of medication and transdermal delivery systems for locally applying drugs. Research has been continuously conducted to efficiently and safely administer pharmaceutical substances such as drugs.

Particularly, as a method for delivering cosmetically effective (skin beautifying) substances to the human body, injecting the effective substances in liquid form through a hypodermic needle is widely applied. Yet, a hypodermic needle with a diameter of several mm may cause pain to a patient by stimulating multiple pain points in the skin, and a high level of skill is required to use the hypodermic needle, which is problematic.

In order to overcome the above disadvantages of the hypodermic needle, methods for transdermal delivery of the above-mentioned effective substances using microneedles having a diameter and height of only tens or hundreds of μm have been actively studied recently. In the case of a microneedle, numerous microneedles may be formed to simultaneously penetrate the stratum corneum, which is the main barrier layer of skin, and by means of the microneedles, a sufficient amount of effective substances is applied to the epidermis layer or dermis layer.

As such, in order to overcome the drawbacks of the injection therapy, research is being conducted on microstructures (microneedles) that are much smaller and less painful than syringe needles. As examples of related art, Korean Patent No. 10-0793615 “BIODEGRADABLE SOLID MICRONEEDLE AND MANUFACTURING METHOD THEREOF”, Korean Patent No. 10-1618523 “MICRONEEDLE AND MICRONEEDLE PATCH”, Korean Patent No. 10-1838715 “COMPOSITION FOR PREPARING MICRONEEDLE CONTAINING CROSS-LINKED HYALURONIC ACID AND NON-CROSS-LINKED HYALURONIC ACID”, and Korean Patent No. 10-2139337 “HYALURONIC ACID FILLER USING MICRONEEDLE PATCH” are known.

However, the aforementioned documents have a problem that a biodegradable polymer microstructure is bent or crushed when penetrating the skin due to its relatively low mechanical strength. Especially, when a polymer derivative having high elasticity is used as a raw material, in manufacturing a microstructure using a molding technique or a drawing technique, there is a limit in that the shape of the desired structure is not created homogeneously, and it is difficult to satisfy the mechanical strength of the microstructure required for skin penetration.

Moreover, in the case of a structure made of hyaluronic acid, the smaller the average molecular weight of hyaluronic acid, the easier it is to form a structure and the lower the viscosity, whereas the larger the average molecular weight of hyaluronic acid, the higher the mechanical strength but the higher the viscosity.

Because of these characteristics, low-molecular hyaluronic acid is generally used as a raw material for microstructures. Still, in the case of microstructure made of low-molecular hyaluronic acid, there is a problem of breaking or bending when penetrating the skin, and it is difficult for cross-linked hyaluronic acid to penetrate the stratum corneum, and a microjet format is difficult to apply due to the viscosity and size of cross-linked hyaluronic acid.

### Documents of Related Art

(Patent Document 0001) Korean Patent No. 10-0793615 “BIODEGRADABLE SOLID MICRONEEDLE AND MANUFACTURING METHOD THEREOF” (Jan. 3, 2008)
(Patent Document 0002) Korean Patent No. 10-1618523 “MICRONEEDLE AND MICRONEEDLE PATCH” (Apr. 28, 2016)
(Patent Document 0003) Korean Patent No. 10-1838715 “COMPOSITION FOR PREPARING MICRONEEDLE CONTAINING CROSS-LINKED HYALURONIC ACID AND NON-CROSS-LINKED HYALURONIC ACID” (Mar. 3, 2018)
(Patent Document 0004) Korean Patent No. 10-2139337 “HYALURONIC ACID FILLER USING MICRONEEDLE PATCH” (Jul. 23, 2020)

### SUMMARY OF THE INVENTION

Accordingly, the present disclosure has been made keeping in mind the above problems occurring in the related art, and the present disclosure is intended to provide a transdermal microneedle patch containing poly-lactic acid microneedles that are easily absorbable into the skin, in which poly-lactic acid (PLA), a biodegradable polymer, is combined with epidermal growth factor (EGF) and hyaluronic acid (HA) to manufacture a non-invasive microneedle adopted as a patch-type filler. According to the present disclosure, since only microneedles are inserted into the skin, use thereof does not require a high degree of skill, and use and convenience are improved. Safety is also improved as there is no secondary infection. In addition, the microneedle patch enables rapid healing with little tissue damage, and is patient-friendly because it does not cause pain. Furthermore, the PLA and EGF elements may be directly delivered into the living tissue after the microneedle patch is applied, and a sufficient amount of PLA and EGF elements may be delivered effectively, stimulating collagen regeneration in skin. Therefore, the application of a filler may be specialized according to cosmetic purposes, and the advantages of a microneedle patch-type filler that delivers cosmetically active substances through the skin may be maximized.

In order to achieve the above objective, according to an embodiment of the present disclosure, there is provided a transdermal microneedle patch containing poly-lactic acid (PLA) microneedles that easily absorbable into skin, the microneedle patch including: at least one needle base disposed to protrude on a surface of the microneedle patch while having a patch layer as a flat layer, and made of hyaluronic acid (HA); and a needle tip disposed on an upper surface of the needle base, separated from the needle base by being inserted into a dermis layer of the skin, and made of the PLA, a biodegradable polymer.

The PLA, a biodegradable polymer, according to the present disclosure may be characterized by after adding 0.01˜0.5 g of epidermal growth factor (EGF) to 0.1˜0.5 g of PLA and dissolving in 10 m [symbol] of dichloromethane (DCM) as an organic solvent, mixing same with 50 m [symbol] of 1 wt % poly vinyl alcohol (PVA) solution, and then mixing the prepared PLA microparticles with a size of 30˜150 μm with 3 wt % carboxylic methyl cellulose (CMC) solution.

In addition, the PLA according to the present disclosure may be poly-L-lactic acid (PLLA), poly-D-lactic acid (PDLA), and poly-D, L-lactide (PDLLA), and may be characterized by using any one of or a mixture of any one or more of the PLLA, PDLA, and PDLLA.

In addition, the needle base and the needle tip according to the present disclosure may be coupled to each other by physical adsorption or adhesion, chemical adsorption, or adhesion.

In addition, a microneedle composed of the needle base and the needle tip according to the present disclosure may be provided in a conical shape with a diameter of 300 μm or less and a length of 300 to 800 μm.

In addition, the microneedle patch according to the present disclosure may be, after applied, rubbed on the skin for 5 to 10 minutes with an output of 0.1 to 5 W/cm2 using ultrasonic waves with a frequency in a range of 1 to 10 MHz to separate the needle tip from the needle base of the microneedle so that the PLA and an EGF may permeate into the dermis layer of the skin.

Meanwhile, terms or words used in this specification and claims should not be construed as being limited to ordinary or dictionary meanings, but should be interpreted as meanings and concepts consistent with the technical spirit of the present disclosure on the basis of the principle that an inventor may properly define the concept of terms in order to best describe his or her invention. Embodiments described in this specification and the configuration shown in the drawings are only the most preferred embodiment of the present disclosure, and does not represent all of the technical spirit of the present disclosure. Therefore, it should be understood that there may be various equivalents and modifications that can replace these embodiments at the time of this application.

As described above in the configuration and operation, according to a transdermal microneedle patch containing PLA microneedles that are easily absorbable into the skin of the present disclosure, poly-lactic acid (PLA), a biodegradable polymer, is combined with epidermal growth factor (EGF) and hyaluronic acid (HA) to manufacture a non-invasive microneedle adopted as a patch-type filler. According to the present disclosure, since only microneedles are inserted into the skin, use thereof t does not require a high degree of skill, and use and convenience are improved. Safety is also improved as there is no secondary infection. In addition, the microneedle patch enables rapid healing with little tissue damage, and is patient-friendly because it does not cause pain. Furthermore, the PLA and EGF elements to be delivered can be directly delivered into the living tissue after the microneedle patch is applied, and a sufficient amount of PLA and EGF elements can be delivered effectively, stimulating collagen regeneration in skin. Therefore, the application of a filler can be specialized according to cosmetic purposes, and the advantages of a microneedle patch-type filler that delivers cosmetically active substances through the skin can be maximized.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a perspective view showing a transdermal microneedle patch containing PLA microneedles that are easily absorbable into the skin according to the present disclosure;

FIG. 2 is a configuration diagram showing a microneedle of FIG. 1 in cross section;

FIGS. 3A and 3B are photographs of the transdermal microneedle patch containing PLA microneedles that are easily absorbable into the skin according to the present disclosure taken with an electron microscope (SEM, JEOL JSM-7500F, 70 times, 80 times);

FIG. 4 is a photograph of the transdermal microneedle patch containing PLA microneedles that are easily absorbable into the skin according to the present disclosure taken with an electron microscope (Nikon Eclipse 80i, 100 times); and

FIGS. 5A to 5C are configuration diagrams showing the process of delivering the PLA element into the skin by attaching the transdermal microneedle patch containing PLA microneedles that are easily absorbable into the skin.

### DETAILED DESCRIPTION OF THE INVENTION

Hereinafter, preferred embodiments according to the present disclosure will be described in detail with reference to the accompanying drawings.

The present disclosure proposes a microneedle and a microneedle patch. Particularly, the present disclosure proposes a transdermal microneedle patch containing poly-lactic acid microneedles that are easily absorbable into the skin, in which poly-lactic acid (PLA), a biodegradable polymer, is combined with epidermal growth factor (EGF) and hyaluronic acid (HA) to manufacture a non-invasive microneedle adopted as a patch-type filler. According to the present disclosure, since only microneedles are inserted into the skin, use thereof does not require a high degree of skill, and use and convenience are improved. Safety is also improved as there is no secondary infection. In addition, the microneedle patch enables rapid healing with little tissue damage, and is patient-friendly because it does not cause pain. Furthermore, the PLA and EGF elements may be directly delivered into the living tissue after the microneedle patch is applied, and a sufficient amount of PLA and EGF elements may be delivered effectively, stimulating collagen regeneration in skin. Therefore, the application of a filler may be specialized according to cosmetic purposes, and the advantages of a microneedle patch-type filler that delivers cosmetically active substances through the skin may be maximized.

Throughout this specification, references are made to patent documents and their citations are indicated. The cited patent documents are the property of the present applicant, the disclosed content of which is incorporated herein by reference, so that the level of the technical field to which the present disclosure pertains and the contents of the present disclosure are more clearly described.

According to the present disclosure, a microneedle patch 1 includes: at least one needle base 10 disposed to protrude on one surface of the microneedle patch while having a patch layer 15 as a flat layer, and made of hyaluronic acid (HA); and a needle tip 20 disposed on the upper surface of the needle base 10, separated from the needle base 10 by being inserted into the dermis layer of skin, and made of poly-lactic acid (PLA), a biodegradable polymer.

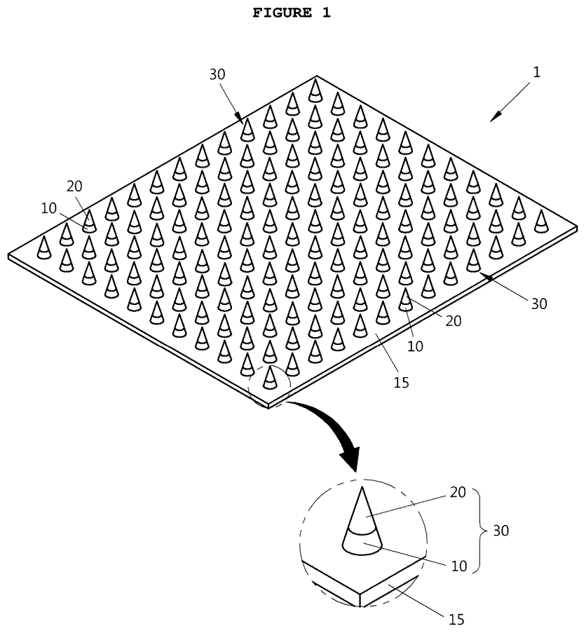

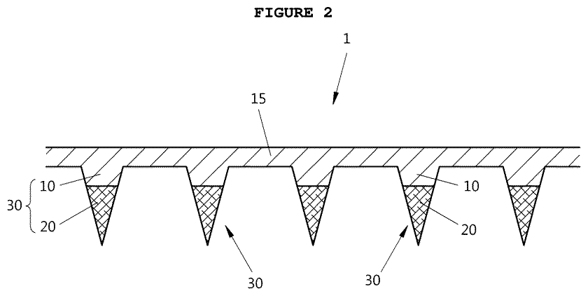

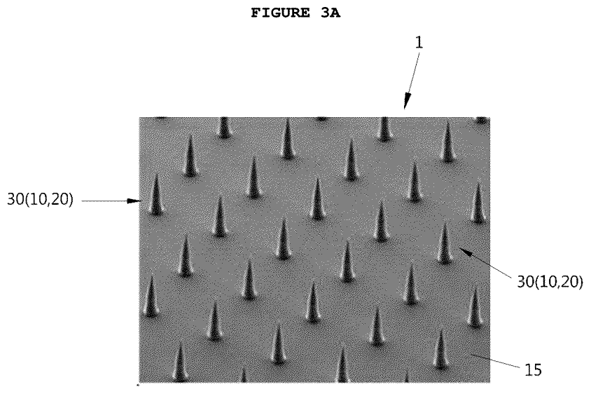

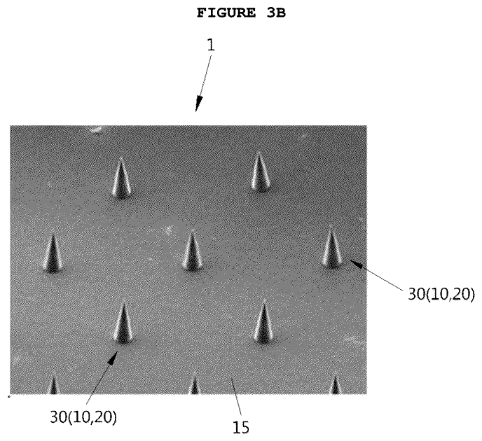

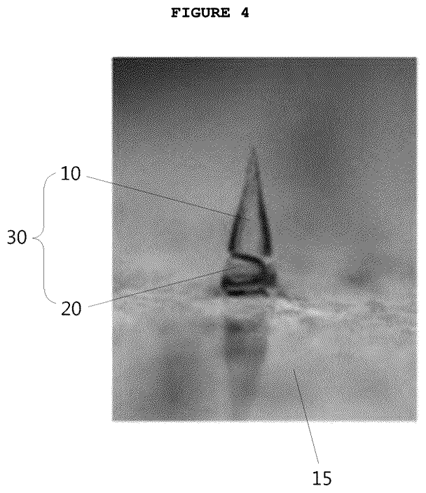

On the other hand, referring to FIGS. 1 to 4, a microneedle patch of the present disclosure is composed of a flat patch layer 15 and a microneedle 30 on the patch layer 15. The microneedle 30 includes a needle base 10 in contact with the patch layer 15 and a needle tip 20 integrally coupled to the needle base 10. Together with the patch layer 15, the needle base 10 is made of hyaluronic acid but is not limited thereto, and biocompatible materials with properties equivalent to hyaluronic acid, that is, biodegradable polymers may also be applied. Unlike the needle base 10, the needle tip 20 is made of poly-lactic acid (PLA), a biodegradable polymer.

The patch layer 15 of the present disclosure is a part that contacts the skin surface without being inserted into the skin when the microneedle patch 1 is applied to the skin. Although not shown in the drawings, the patch layer 15 may be provided with an adhesive film that is easily removed when the microneedle patch 1 is used.

According to the detailed configuration of the present disclosure, the biodegradable polymer poly-lactic acid (PLA) is characterized by after adding 0.01˜0.5 g of epidermal growth factor (EGF) to 0.1˜0.5 g of PLA and dissolving in 10 m [symbol] of dichloromethane (DCM) as an organic solvent, mixing same with 50 m [symbol] of 1 wt % poly vinyl alcohol (PVA) solution, and then mixing the prepared PLA microparticles with a size of 30˜150 μm with 3 wt % carboxylic methyl cellulose (CMC) solution. The poly-lactic acid (PLA) of the present disclosure is prepared on the basis of Korean Patent No. 10-1854540, which is the registered right of the present disclosure well-known in the art, “MANUFACTURING METHOD OF PLLA DERMAL FILLER WITH EXCELLENT DURABILITY” (Apr. 26, 2018), but is not limited thereto.

Simply described, oil is prepared by dissolving 10 m [symbol] of an organic solvent in 0.1˜0.5 g of PLA, biodegradable polymer, adding 50 m [symbol] of 1 wt % poly vinyl alcohol (PVA) solution to the total amount of the oil and stirring for one hour to mix homogeneous PLA to maintain an oil-in-water emulsion state. Then, 200 m [symbol] of distilled water is mixed with the oil-in-water emulsion and evaporated by stirring for two hours, and then the oil-in-water emulsion from which the organic solvent is evaporated is dried for 24 hours using a freeze dryer at −50° C. to produce PLA made of microparticles.

Next, after mixing 0.01˜0.5 g of epidermal growth factor (EGF) with poly-lactic acid (PLA) made of microparticles, 10 m [symbol] of an organic solvent is dissolved to prepare oil, and then 50 m [symbol] of 1 wt % poly vinyl alcohol (PVA) solution is added to the total amount of the oil and stirred for one hour to maintain an oil-in-water emulsion state mixed with homogeneous PLA. Then, 200 m [symbol] of distilled water is mixed with the oil-in-water emulsion and stirred for two hours to evaporate the organic solvent, and then the oil-in-water emulsion from which the organic solvent is evaporated is dried for 24 hours using a freeze dryer at −50° C. to produce microparticle PLA mixed with EGF. The final selected microparticle PLA mixed with EGF with a size of 30˜150 μm is mixed with 3 wt % carboxylic methyl cellulose (CMC) solution to make a suspension to be used as the needle tip 20 constituting the microneedle 30.

In addition, the poly-lactic acid (PLA) according to the present disclosure is poly-L-lactic acid (PLLA), poly-D-lactic acid (PDLA), and poly-D, L-lactide (PDLLA), and is characterized by using any one of or a mixture of any one or more of PLLA, PDLA, and PDLLA. Poly-lactic acid (PLA) is an aliphatic polyester, and a thermoplastic polymer material synthesized using monomers obtained from 100% renewable resources such as corn and potato starch. Such PLA has three stereoisomers: PLLA, PDLA, and PDLLA. The PLA used in the microneedle 30 refers to PLLA, PDLA, and PDLLA, and any one of or a mixture of any one or more of PLLA, PDLA, and PDLLA is used.

Growth factor EGF is also known as epidermal proliferation factor, epithelial growth factor, or epithelial proliferation factor. EGF is a protein derived from epithelial or integumentary cells and acts as a kind of ligand and binds to epidermal growth factor receptor (EGFR), contributing to cell growth, differentiation, and glycolysis, and being used for skin transplantation and promoting wound healing. EGF is a representative growth factor, but it is not limited to, and all nutrients that can benefit the skin are applicable.

In addition, according to the detailed configuration of the present disclosure, the needle base 10 and the needle tip are coupled to each other by physical adsorption or adhesion, chemical adsorption, or adhesion, and the microneedle 30 composed of the needle base 10 and the needle tip 20 is provided in a conical shape with a diameter of 300 μm or less and a length of 300 to 800 μm. The needle base 10 and the needle tip 20 constituting the microneedle 30 bonded to each other by physical adsorption or adhesion, chemical adsorption, or adhesion, and preferably the bonding may be achieved through a low-temperature process by heat. The needle base 10 and the needle tip 20 made of different ingredients are manufactured by a micro molding method or a droplet born air blowing (DAB) method. The microneedle 30 is provided in a conical shape with a diameter of 300 μm or less and with a length, that is, a height (sum of the needle base 10 and the needle tip 20) of 300 to 800 μm without limitation.

In addition, the microneedle patch 1 according to the present disclosure is characterized in that after the patch 1 is applied to the skin, the patch 1 is rubbed on the skin for 5 to 10 minutes with an output of 0.1 to 5 W/cm2 using ultrasonic waves with a frequency in the range of 1 to 10 MHz to separate the needle tip 20 from the needle base 10 of the microneedle 30 so that the PLA and EGF elements permeate into the dermis layer in the skin. For the microneedle patch 1, the needle tip 20 made of the poly-lactic acid (PLA) element and the epidermal growth factor (EGF) element in the skin of the subject is separated from the needle base 10 made of hyaluronic acid (HA) by using ultrasonic waves, and HA has a light molecular weight, whereas PLA has a heavy molecular weight. Due to this, when ultrasonic waves are applied, the needle base 10 having a light molecular weight is separated from the needle tip 20.

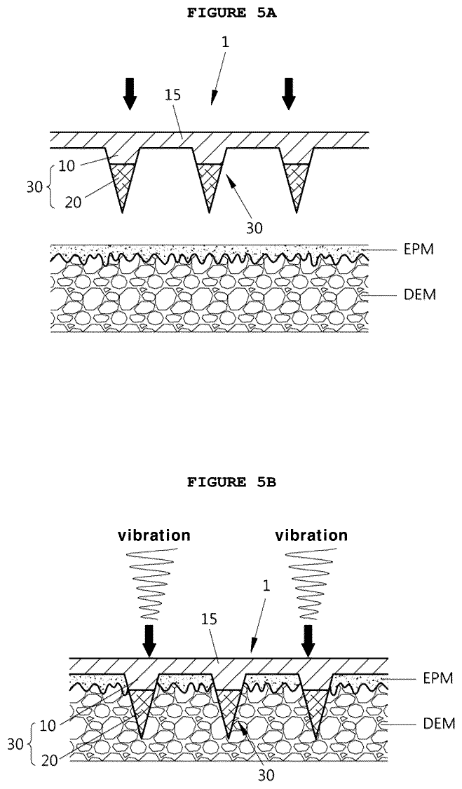

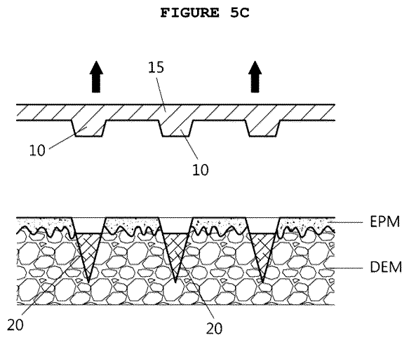

Referring to FIGS. 5A to 5C, when the microneedle patch 1 is applied to the skin, the patch 1 is attached to the skin by pressing it by hand, or the patch 1 is attached to the skin using a dedicated applicator. At this time, the needle tip 20 is inserted into the dermis. Then, the microneedle patch 1 is rubbed on the skin for 5 to 10 minutes with an output of 0.1 to W/cm2 using ultrasonic waves with a frequency in the range of 1 to 10 MHz, and when the patch 1 is removed, the needle base is separated from the needle tip 20 and naturally, the needle tip 20 remains being inserted into the dermis so that the PLA element and the growth factor EGF element permeate into the dermis layer in the skin. Therefore, the PLA and EGF elements may be easily delivered directly into living tissue, and a sufficient amount of PLA and EGF elements may be delivered effectively, stimulating collagen regeneration in skin, and the application of a filler may be specialized according to cosmetic purposes.

As such, in the present disclosure, poly-lactic acid (PLA), a biodegradable polymer, is combined with epidermal growth factor (EGF) and hyaluronic acid (HA) to manufacture a non-invasive microneedle adopted as a patch-type filler. According to the present disclosure, since only microneedles are inserted into the skin, use thereof does not require a high degree of skill, and use and convenience are improved. Safety is also improved as there is no secondary infection. In addition, the microneedle patch enables rapid healing with little tissue damage, and is patient-friendly because it does not cause pain. Furthermore, the PLA and EGF elements may be directly delivered into the living tissue after the microneedle patch is applied, and a sufficient amount of PLA and EGF elements may be delivered effectively, stimulating collagen regeneration in skin. Therefore, the application of a filler may be specialized according to cosmetic purposes, and the advantages of a microneedle patch-type filler that delivers cosmetically active substances through the skin may be maximized.

The present disclosure is not limited to the described embodiments, and it should be obvious to those skilled in the art that various modifications and variations may be made without departing from the spirit and scope of the present disclosure.

What is claimed is:

1. A transdermal microneedle patch containing poly-lactic acid (PLA) microneedles that are easily absorbable into skin, the microneedle patch comprising:
at least one needle base disposed to protrude on a surface of the microneedle patch while having a patch layer as a flat layer, and made of hyaluronic acid (HA); and
a needle tip disposed on an upper surface of the needle base, separated from the needle base by being inserted into a dermis layer of the skin, and made of the PLA, a biodegradable polymer.

2. The microneedle patch of claim 1, wherein the PLA, a biodegradable polymer, is characterized by after adding 0.01˜0.5 g of epidermal growth factor (EGF) to 0.1˜0.5 g of PLA and dissolving in 10 m [symbol] of dichloromethane (DCM) as an organic solvent, mixing same with 50 m [symbol] of 1 wt % poly vinyl alcohol (PVA) solution, and then mixing the prepared PLA microparticles with a size of 30˜150 μm with 3 wt % carboxylic methyl cellulose (CMC) solution.

3. The microneedle patch of claim 1, wherein the PLA is poly-L-lactic acid (PLLA), poly-D-lactic acid (PDLA), and poly-D, L-lactide (PDLLA), and is characterized by using any one of or a mixture of any one or more of the PLLA, PDLA, and PDLLA.

4. The microneedle patch of claim 1, wherein the needle base and the needle tip are coupled to each other by physical adsorption or adhesion, chemical adsorption, or adhesion.

5. The microneedle patch of claim 1, wherein a microneedle composed of the needle base and the needle tip is provided in a conical shape with a diameter of 300 μm or less and a length of 300 to 800 μm.

6. The microneedle patch of claim 1, wherein the microneedle patch is, after applied, rubbed on the skin for 5 to 10 minutes with an output of 0.1 to 5 W/cm2 using ultrasonic waves with a frequency in a range of 1 to 10 MHz to separate the needle tip from the needle base of the microneedle so that the PLA and an EGF permeate into the dermis layer in the skin.
